(19) United States
(12) Patent Application Publication
(10) Pub. No.: US 2026/0054255 A1
(43) Pub. Date: Feb. 26, 2026

(54) PHOTOCATALYTIC CO2 REDUCTION WITH BINARY CATALYST-DECORATED NANOSTRUCTURES

(71) Applicants: Pengfei Ou, Ann Arbor, MI (US); Jun Song, Ann Arbor, MI (US); The Regents of the University of Michigan, Ann Arbor, MI (US)

(72) Inventors: Zetian Mi, Ann Arbor, MI (US); Pengfei Ou, Montreal (CA); Zhengwei Ye, Ann Arbor, MI (US); Yongjin Ma, Ann Arbor, MI (US); Jun Song, Montreal (CA); Baowen Zhou, Ann Arbor, MI (US)

(21) Appl. No.: 18/770,020

(22) Filed: Jul. 11, 2024

Related U.S. Application Data
(60) Provisional application No. 63/526,117, filed on Jul. 11, 2023.

(51) Int. Cl. B01J 23/52; B01J 19/12; B01J 35/39; C07C 1/02; C09K 11/08; C09K 11/62; C23C 18/14; C30B 23/04; C30B 29/40; C30B 29/60

(52) U.S. Cl. CPC B01J 23/52; B01J 19/127; B01J 35/39; C07C 1/02; C09K 11/0883; C09K 11/62; C23C 18/143; C30B 23/04; C30B 29/403; C30B 29/406; C30B 29/605; B01J 2219/0875; B01J 2219/0892; B01J 2219/1203; C07C 2523/46; C07C 2523/52

(57) ABSTRACT

A photocatalytic device includes a substrate and an array of conductive projections supported by the substrate and extending outward from the substrate. Each conductive projection of the array of conductive projections has a semiconductor composition configured for charge carrier generation in response to light radiation. Each conductive projection of the array of conductive projections is decorated with a catalyst arrangement, the catalyst arrangement including a parental Group IB metal and a secondary platinum group metal.

### CROSS-REFERENCE TO RELATED APPLICATION

This application claims the benefit of U.S. provisional application entitled “Photocatalytic CO₂ Reduction With Binary Catalyst-Decorated Nanostructures,” filed Jul. 11, 2023, and assigned Ser. No. 63/526,117, the entire disclosure of which is hereby expressly incorporated by reference.

### STATEMENT REGARDING FEDERALLY SPONSORED RESEARCH OR DEVELOPMENT

This invention was made with government support under Contract No. W911NF-21-1-0337 awarded by the U.S. Army Research Office. The government has certain rights in the invention.

### BACKGROUND OF THE DISCLOSURE

### Field of the Disclosure

The disclosure relates generally to photocatalytic reduction of carbon dioxide (CO₂).

### Brief Description of Related Technology

Recycling of carbon dioxide (CO₂) toward clean fuels and chemicals offers an attractive pathway to address the critical issues of energy shortage and climate change. Compared with electrocatalysis, thermocatalysis, and biocatalysis that involve extra heat/electricity input and/or complex setups, photocatalysis has emerged as a promising carbon fixation technology because of its simple configuration, low cost, and environmental accountability.

Photocatalytic synthesis of C₂₊ compounds without sacrificial agents is particularly interesting but has remained extremely challenging. Thus far, a broad range of photocatalytic devices have been explored for CO₂ reduction reactions by assembling various semiconductors with suitable catalysts. However, state-of-the-art photocatalytic devices predominantly yield C₁ products with low activity in the order of micromoles per gram catalysts per hour (μmol g⁻¹ h⁻¹) and low light-to-fuels (LTFs) energy efficiency of less than 0.1%. Meanwhile, sacrificial agents were essentially required in these studies.

The drawbacks to previous attempts at photocatalytic synthesis of C₂₊ compounds may be attributed to the following reasons. For most of the semiconductors, it is difficult to provide sufficient redox potentials without compromising light absorption, due to the fixed band structures. Moreover, there is a lack of an efficient electron-migration channel in the systems, suffering from severe electron-hole recombination. Most significantly, C—C coupling is a highly endergonic process with sluggish kinetics, remaining as a fundamental bottleneck of applied bias-free C₂₊ compound synthesis.

Known CO₂ reduction catalysts include enzymes, molecular catalysts, and metal/metal oxides. Among various materials, copper (Cu) and its derivatives are well recognized to be efficient catalysts capable of synthesizing C₂₊ compounds because of their unique properties. Through defect engineering, surface reconstruction, and oxidation states tuning, a broad range of C₂₊ compounds including C₂H₄ and C₂H₅OH have been produced over copper-based catalysts. However, large overpotentials are in principle required to drive the reactions by electrocatalysis.

It is thus been challenging to directly synthesize C₂₊ compounds from CO₂ and H₂O by photocatalysis, which is highly attractive for practical applications compared to that conducted with applied bias and/or sacrificial agents.

### SUMMARY OF THE DISCLOSURE

In accordance with one aspect of the disclosure, a photocatalytic device includes a substrate and an array of conductive projections supported by the substrate and extending outward from the substrate, each conductive projection of the array of conductive projections having a semiconductor composition configured for charge carrier generation in response to light radiation. Each conductive projection of the array of conductive projections is decorated with a catalyst arrangement, the catalyst arrangement including a parental Group IB metal and a secondary platinum group metal.

In accordance with another aspect of the disclosure, a method of fabricating a photocatalytic device includes providing a substrate having a surface, forming an array of conductive projections on the substrate such that each conductive projection of the array of conductive projections extends outward from the substrate, each conductive projection of the array of conductive projections having a semiconductor composition configured for charge carrier generation in response to light radiation, and decorating each conductive projection of the array of conductive projections with a catalyst arrangement. Decorating each conductive projection includes concurrently depositing a parental Group IB metal and a secondary platinum group metal.

In accordance with yet another aspect of the disclosure, a catalytic device includes a substrate and a catalyst arrangement supported by the substrate, the catalyst arrangement establishing a nitride/catalyst interface of the catalytic device. The catalyst arrangement includes a parental Group IB metal and a secondary platinum group metal.

In accordance with still yet another aspect of the disclosure, a catalytic device includes a substrate and a binary catalyst arrangement supported by the substrate and configured for CO2 reduction. The binary catalyst arrangement includes gold and iridium.

In connection with any one of the aforementioned aspects, the devices and/or methods described herein may alternatively or additionally include or involve any combination of one or more of the following aspects or features. The parental Group IB metal is gold. The secondary platinum group metal is iridium. The catalyst arrangement is a binary catalyst arrangement. The catalyst arrangement includes an alloy of gold and iridium. The catalyst arrangement has a parental-to-secondary metal ratio that falls in a range about 0.45/0.55 to about 0.75/0.25. The semiconductor composition includes a III-nitride semiconductor material. The III-nitride semiconductor material is InGaN. Each conductive projection of the array of conductive projections includes a nanowire. Each conductive projection of the array of conductive projections includes a layered arrangement of semiconductor materials. The layered arrangement of semiconductor materials establishes a multiple band structure. The catalyst arrangement is configured for catalysis of carbon dioxide (CO₂) reduction. A method of using a photocatalytic device as described herein includes illuminating the photocatalytic device with incident light radiation, and capturing a product of the CO₂ reduction. The catalyst arrangement is configured such that the product includes C₂H₆. The photocatalytic device is illuminated without application of a bias voltage to the photocatalytic device. The method further includes disposing the photocatalytic device in a container and supplying water or water vapor and CO₂ to the container. Illuminating the photocatalytic device is implemented while the container is free of a sacrificial agent for the CO₂ reduction. Decorating each conductive projection includes configuring a deposition procedure to establish a parental metal-to-secondary metal ratio that falls in a range about 0.45/0.55 to about 0.75/0.25. Concurrently depositing the parental Group IB metal and the secondary platinum group metal includes implementing a photo-deposition procedure. Forming the array of conductive projections includes implementing a molecular beam epitaxy (MBE) procedure to grow a stack of a plurality of III-nitride semiconductor segments. Each III-nitride semiconductor segment of the plurality of III-nitride semiconductor segments has a respective bandgap for charge carrier generation in response to solar radiation. The stack includes a plurality of GaN segments, each GaN segment of the plurality of GaN segments being disposed between a respective adjacent pair of III-nitride semiconductor segments of the plurality of III-nitride semiconductor segments. The catalytic device further includes a structure supported by the substrate, the structure including a nitride surface, the nitride surface being decorated with the binary catalyst arrangement. The binary catalyst arrangement is configured to catalyze CO₂ conversion into multi-carbon products. A system includes a catalytic device as described herein and a thermochemical cell in which the catalytic device is disposed. A system includes a catalytic device as described herein and an electrochemical cell in which the catalytic device is disposed.

### BRIEF DESCRIPTION OF THE DRAWING FIGURES

For a more complete understanding of the disclosure, reference should be made to the following detailed description and accompanying drawing figures, in which like reference numerals identify like elements in the figures.

FIG. 1 is a schematic view of a photocatalytic device having a binary catalyst arrangement for reduction of carbon dioxide (CO₂) in accordance with one example.

FIG. 2 is a flow diagram of a method of fabricating a photocatalytic device having a binary catalyst arrangement for reduction of carbon dioxide (CO₂) in accordance with one example.

FIG. 3 depicts structural, chemical, and physical property characterization of InGaN nanowires decorated with various cocatalysts, including (A) a 45° tilted-SEM image and energy band structure of InGaN nanowires on Si, (B) HAADF-STEM and elemental mapping images of pure Au nanoparticles on InGaN nanowires, (C) XRD spectra of InGaN nanowires on Si, Au nanoparticles on InGaN nanowires, and AuIr decorated InGaN nanowires on Si, (D) HAADF-STEM and elemental mapping images of a device having AuIr decorated InGaN nanowires on Si in accordance with one example, (E) high-resolution XPS spectra of Au 4f, (F) temperature-dependent photoluminescence spectroscopy of InGaN nanowires on Si, and (G) transient reflection spectroscopy of InGaN nanowires, Au nanoparticles on InGaN nanowires, and the example device having AuIr decorated InGaN nanowires on Si.

FIG. 4 depicts graphical plots of (A) evolution rate and (B) selectivity of different products from CO₂ reduction over pure Au nanoparticles on InGaN nanowires with various amounts of Au, (C) time course of CO and CH₄ evolved from CO₂ reduction over Au₂ decorated InGaN nanowires, (D) evolution rate and (E) selectivity, and (F) LTF efficiencies of various products from CO₂ reduction over an example device having AuIr decorated InGaN nanowires on Si with various Au/Ir ratios (reaction conditions: 30 mL distilled water, CO₂, 300 W Xenon lamp, 3.5 W cm⁻²).

FIG. 5 depicts graphical plots of turnover frequency (TOF) and yield of C₂H₆ (A, D), CH₄ (B, E), and CO (C, F) from CO₂ reduction over a device having Au_{0.44}Ir_{0.56} decorated InGaN nanowires on Si in water in accordance with one example (reaction conditions: 30 mL distilled water, CO₂, 300 W Xenon lamp, 3.5 W cm⁻²).

FIG. 6 depicts graphical plots of (A) TOF of C₂H₆ of various feedstocks in water over a device having Au_{0.44}Ir_{0.56} decorated InGaN nanowires on Si in accordance with one example (reaction conditions: 30 mL distilled water, 300 W Xenon lamp, 3.5 W cm⁻²), (B) DRIFTS spectra recorded from CO₂ reduction over the Au_{0.44}Ir_{0.56} decorated InGaN/Si example device, (C) calculated reaction energy (orange column) and energy barrier (blue column) of different C—C coupling mechanism on Au₂Ir₂(111) facet, including *CH+*CH→*C₂H₂, *CH₂+*CH₂→*C₂H₄, *CH₃+*CH₃→C₂H₆(g), and CO₂ insertion into *CH₃, i.e., *CH₃+CO₂(g)→*CH₃COO, and (D) a free energy diagram of the reaction pathway before C—C coupling on Au₂Ir₂(111) facet, as well as (E) a schematic depiction of the operation of the example device, in which yellow, blue, red, grey, and white spheres (labeled Au, Ir, O, C, and H, respectively) represent the atoms of gold, iridium, oxygen, carbon, and hydrogen, respectively.

The embodiments of the disclosed devices and methods may assume various forms. Specific embodiments are illustrated in the drawing and hereafter described with the understanding that the disclosure is intended to be illustrative. The disclosure is not intended to limit the invention to the specific embodiments described and illustrated herein.

### DETAILED DESCRIPTION OF THE DISCLOSURE

Photocatalytic and other devices having conductive projections decorated with a catalyst arrangement (e.g., binary catalyst arrangement) for CO₂ reduction are described. The binary or other catalyst arrangement includes a plurality of nanoparticles disposed on the conductive projections. As described below, each nanoparticle includes a Group IB parental metal, such as gold, and a secondary platinum group metal, such as iridium. The combination provided by the disclosed devices of an effective catalyst arrangement coupled with an effective semiconductor light absorber addresses the challenges that have not been met in previous attempts to break the bottleneck of bias-free C₂₊ compound synthesis. Methods for fabricating such catalytic devices are also described.

The disclosed devices are capable of catalyzing the reduction of CO₂ with water in the absence of applied bias and/or sacrificial agents. For instance, triethanolamine or other sacrificial agents are not required to close the reaction. In this way, operation of the devices may only involve water, CO₂, and sunlight as inputs. As a result, the production of ethane (C₂H₆), other C₂₊ compounds, and/or other chemicals may be realized.

As described herein, the binary catalyst arrangement may include gold regulated, manipulated, or otherwise mediated by iridium. Although described in connection with examples having a binary catalyst arrangement of AuIr, the disclosed devices are not limited to use of Au as the parental metal or the use of Ir as the secondary metal. For instance, additional or alternative Group IB metals, such as Ag and Cu, may be used as the parental metal. Additional or alternative platinum group metals, such as ruthenium, rhodium, palladium, osmium, and platinum may be used as the secondary metal.

III-nitride materials have emerged as a useful family of semiconductor materials for artificial photosynthesis owing to their convenient structural, optical, and electronic properties. It is noted that by state-of-the-art molecular beam epitaxy technology, III-nitrides have been successfully applied to a variety of process including overall water splitting and CO₂ reduction toward C₁ products, e.g., CO and CH₄ by photocatalysis, which is distinct from conventional semiconductors. III-nitride materials may thus be useful in constructing an artificial photosynthesis integrated device (APID) by coupling a rationally designed catalyst for applied bias-free synthesis of C₂₊ compounds from CO₂ and H₂O.

The disclosed devices may include nanowires, nanostructures, or other conductive projections that include one or more III-nitride semiconductors configured for charge carrier generation in response to solar radiation. For instance, each nanowire, nanostructure, or other conductive projection may include multiple segments with differing alloy concentrations to capture multiple bands of solar wavelengths. Each nanowire, nanostructure, or other conductive projection is decorated with a binary catalyst arrangement as described herein. In other cases, the binary catalyst arrangement may be supported by a substrate in other manners, either directly or indirectly.

In a number of the disclosed devices, gold is used as the parental metal and coupled with iridium as the secondary metal for catalyzing CO₂ reduction to C2 products because of its weak interactions with reactants/intermediates. Thus, in such cases, C—C coupling is achieved by inserting CO₂ into the —CH3 intermediate over AuIr. Benefiting from the distinct properties of one-dimensional (“1-D”) InGaN nanowires, the assembly of AuIr with 1-D InGaN nanowires demonstrates applied bias-free synthesis of C₂₊ compounds from CO₂ and H₂O without sacrificial agents by photocatalysis.

The epitaxial growth of 1-D InGaN nanowires may be implemented as set forth in the following publications: Kibria, M. et al., “Visible light-driven efficient overall water splitting using p-type metal-nitride nanowire arrays,” Nat. Commun. 6, 1-8 (2015); Wang, D. et al., “Wafer-level photocatalytic water splitting on GaN nanowire arrays grown by molecular beam epitaxy,” Nano Lett. 11, 2353-2357 (2011), Guan, X. et al., “Making of an industry-friendly artificial photosynthesis device,” ACS Energy Lett. 3, 2230-2231 (2018), U.S. Patent Publication No. 2023/0017032 (“CO₂ Conversion with Metal Sulfide Nanoparticles”), and International Application No. PCT/US23/24569 (“Photocatalytic CO₂ Reduction with Co-Catalyst-Decorated Nanostructures,” published as WO 2023/239712), the entire disclosures of which are hereby incorporated by reference.

Examples of nanowire structures integrated with AuIr binary catalysts for CO₂ photoreduction to C₂ products are provided below. Collectively, a C₂H₆ activity of 58.8 mmol g⁻¹ h⁻¹ was achieved under concentrated illumination with an appreciable total selectivity of about 17.6% for CH₄ and C₂H₆. Taking the useful byproducts of syngas into consideration, the example device exhibited a LTFs efficiency of about 0.59%, with an impressive turnover number of 54,595 for C₂H₆ without obvious performance degradation over 60 hours. This example established an effective APID for C₂₊ compounds synthesis using light, carbon dioxide, and water as the only inputs.

The III-nitride semiconductor-based projections and binary catalyst arrangement present a useful combination of an effective catalyst and a semiconductor platform for the CO₂ reduction reaction. The III-nitride semiconductor materials are highly efficient in generating charge carriers from solar radiation. The III-nitride semiconductor-based projections also provide a nitride surface at which the binary catalyst arrangement is disposed. The binary catalyst arrangement thus establishes a nitride/catalyst interface of the disclosed catalytic devices. The nature of the nitride/catalyst interface may vary from the examples described below. For instance, other structures may be used to provide the nitride surface. The characteristics of the nitride surface may also vary. For instance, the nitride surface may be composed of, or otherwise include, an oxynitride material.

The disclosed devices and systems may include multi-band (e.g., quadruple-band) for artificial photosynthesis and solar fuel conversion with significantly improved performance. For instance, the disclosed devices and systems may include InGaN nanowire arrays to improve the efficiency of the conversion. For example, each nanowire may include layers or segments of different semiconductor compositions, such as In_{0.35}Ga_{0.65}N, In_{0.27}Ga_{0.73}N, In_{0.20}Ga_{0.80}N, and GaN, which present energy bandgaps about 2.1 eV, 2.4 eV, 2.6 eV, and 3.4 eV, respectively. As described herein, such multi-band InGaN and other nanowire arrays are integrated directly on a wafer for enhanced light absorption. Each nanostructure may thus be capable of absorbing a wide range of the solar spectra, including, for instance, ultraviolet and visible portions of the solar spectra.

The configuration of the multi-band nanostructure arrays may vary. For instance, additional, fewer, or alternative segments may be included.

Although described herein in connection with electrodes having GaN-based nanowire arrays for CO₂ reduction, the disclosed devices are not limited to GaN-based nanowire arrays. A wide variety of other types of nanostructures and other conductive projections may be used. Other III-nitride semiconductors may also be used. Thus, the nature, construction, composition, configuration, characteristics, shape, and other aspects of the conductive projections through which the CO₂ reduction is catalyzed may vary.

In some cases, the disclosed devices are configured for CH₄, C₂H₆ and/or other C—C compound synthesis using a binary catalyst arrangement (e.g., Au and Ir) in coordination with multi-stacked (or multi-band) InGaN/GaN nanowires. This combination allows ethane to be produced solely from CO₂, H₂O, and solar light. The binary catalysts work synergistically to select the syntheses of the C—C compound(s). Examples that combine the optoelectronic properties of the multi-stacked InGaN/GaN nanowires with the binary catalyst arrangement are described below.

Although described in connection CO₂ reduction into CH₄ and C₂H₆, the disclosed photocatalytic devices and systems may be used in other chemical reaction contexts and applications. For instance, the disclosed photocatalytic devices and systems may be useful in connection with CO₂ reduction to various fuels and other chemicals, such as C₂H₄ and C₂H₅OH. The photocatalytic devices may also be used in connection with still other reactions not involving CO₂ reduction, such as nitrogen reduction to ammonia.

Although described herein in connection with photocatalytic devices, one or more aspects of the disclosed devices may be applied to catalyzing other types of reactions. For instance, the disclosed devices may be used to catalyze reactions driven by electrical or thermal energy (either alone or in combination and/or in combination with light). In some cases, the disclosed devices may also be used in connection with reactions involving the application of a bias voltage and/or sacrificial agents.

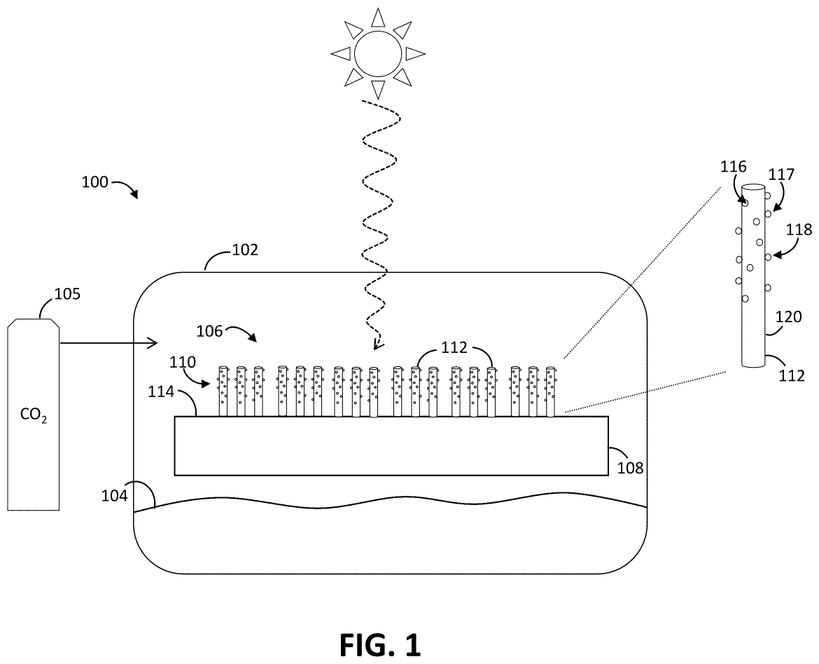

FIG. 1 depicts a photocatalytic system 100 for CO₂ reduction. The CO₂ reduction may include or involve photocatalytic water splitting. Other chemical reactions may also be implemented or supported by the system 100. In this example, the photocatalytic system 100 includes a container 102. In some cases, the container 102 is configured as a sealed reactor, such as a sealed gas-phase reactor. The container 102 may be configured to allow illumination (e.g., solar illumination) of the interior of the container 102. For instance, the container 102 may have a transparent cover, side, cap, or other portion, such as a quartz top. The manner in which the system 100 is illuminated may vary. The size, construction, composition, configuration, and other characteristics of the container 102 may vary. The system 100 may not include a container in other cases.

In the example shown, liquid water 104 is disposed in the container 102. The water 104 may or may not be pure water (e.g., distilled water). The pH of the water 104 may vary accordingly. In some cases, the water 104 evaporates and/or is vaporized prior to operation. Alternatively or additionally, water vapor may be provided to the container directly. In still other cases, all of the water 104 remains in the liquid phase.

The system 100 may include a source 105 of CO₂ coupled to the container 102. The CO₂ source 105 may be integrated to any desired extent with a source of water or water vapor. In some cases, the system 100 receives CO₂ passively and/or without an express source. For example, CO₂ may be supplied in part or whole from the ambient.

As described herein, the system 100 does not include a voltage or other source of electrical energy. Thus, in the example of FIG. 1, the system 100 accordingly implements the CO₂ reduction without the application of a bias voltage to a photocatalytic device of the system 100. In other cases, one or more bias voltages may be applied to one or more electrodes or other components in the system 100.

The system 100 may also be free of sacrificial agents. In the example of FIG. 1, the container 102 is sacrificial agent-free. In other cases, one or more sacrificial agents may be used to promote the CO₂ reduction reaction in the system 100.

The photocatalytic system 100 includes a photocatalytic device 106 disposed in the container 102. The photocatalytic device 106 may or may not be immersed (e.g., partially or completely) in the water 104. In the example of FIG. 1, the photocatalytic device 106 is disposed in the container 102 in a manner to allow the incident light to illuminate the semiconductor device 106. In some cases, the photocatalytic device 106 is configured for CO₂ reduction with water splitting in response to the illumination.

The semiconductor device 106 includes a substrate 108 and an array 110 of conductive projections 112 supported by the substrate 108. In some cases, each conductive projection 112 is or includes a nanowire or other nanostructure. In this example, each conductive structure 112 is or includes a cylindrically shaped nanostructure. The cylindrical shape has a circular cross-sectional shape (e.g., a circular cylinder), as opposed to, for instance, a plate-shaped or sheet-shaped nanostructure. The conductive projections 112 may thus be configured, and/or referred to herein, as nanowires. In this example, the nanowires 112 extend outward from a top or upper surface 114 of the substrate 108. Alternative or additional surfaces of the substrate 108 may support the array 110.

The substrate 108 may be active (e.g., functional) and/or passive (e.g., structural). In one example of the former case, the substrate 108 may be or include a reflective material or layer to direct light back toward the nanowires 112. In one example of the latter case, the substrate 108 may be configured and act solely as a support structure for the nanowires 112. Alternatively or additionally, the substrate 108 may be composed of, or otherwise include, a material suitable for the growth or other deposition of the nanowires 112.

The substrate 108 may include a light absorbing material. In such cases, the light absorbing material is configured to generate charge carriers upon solar or other illumination. The light absorbing material has a bandgap such that incident light generates charge carriers (electron-hole pairs) within the substrate. Some or all of the substrate 108 may be configured for photogeneration of electron-hole pairs.

The substrate 108 may include a semiconductor material. In some cases, the substrate 108 is composed of, or otherwise includes, silicon. For instance, the substrate 108 may be provided as a silicon wafer. The silicon may or may not be doped. The doping arrangement may vary. For example, one or more components of the substrate 108 may be non-doped (intrinsic), or effectively non-doped. The substrate 108 may include alternative or additional layers, including, for instance, support or other structural layers. The composition of the substrate 108 may thus vary. For example, the substrate may be composed of, or otherwise include, metal films, GaAs, GaN, or SiOₓ in other cases.

The substrate 108 may establish a surface, e.g., the surface 114, at which a catalyst arrangement (e.g., a photocatalyst arrangement) of the semiconductor device 106 is provided. The photocatalyst arrangement is provided and supported by the nanowires 112 of the array 110. The photocatalyst arrangement may include a binary catalyst arrangement having a parental Group IB metal and a secondary platinum group metal as described herein.

Each nanowire 112 has a semiconductor composition for charge carrier generation in response to solar radiation. In some cases, the semiconductor composition includes one or more III-nitride semiconductor materials, such as gallium nitride (GaN) and/or one or more alloys of indium gallium nitride (InGaN). Further details regarding examples having stacks of GaN/InGaN segments are provided below. Additional or alternative semiconductor materials may be used, including, for instance, indium nitride, indium gallium nitride, aluminum nitride, boron nitride, aluminum oxide, and silicon, gallium phosphide, gallium arsenide, indium phosphide, tantalum nitride, silicon, and other semiconductor materials.

Each nanowire 112 may be or include a columnar, rod-shaped, post-shaped, or other elongated structure. The nanowires 112 may be grown or formed as described in U.S. Pat. No. 8,563,395 (“Method of growing uniform semiconductor nanowires without foreign metal catalyst and devices thereof”), the entire disclosure of which is hereby incorporated by reference. The dimensions (e.g., length, diameter), size, shape, and other characteristics of the nanowires 112 may vary.

The semiconductor composition of each nanowire 112 allows charge carriers to be generated to support the CO₂ reduction reaction and water splitting (i.e., water oxidation reaction of 2H₂O->O₂+4H⁺+4e⁻). Proton diffusion from the water oxidation reaction to the CO₂ reduction reaction may occur across a single one of the nanowires 112. Alternatively or additionally, the proton diffusion may occur between two adjacent nanowires 112 in the array 110. The protons may diffuse through liquid water present between the nanowires 112 and/or through the water 104 or other liquid in which the device 100 is immersed.

Each nanowire 112 extends outward from the surface 114 of the substrate 108. In this example, the surface 114 of the substrate 108 is planar. Alternatively or additionally, the surface 114 of the substrate is nonplanar. In such cases, one or more subsets of the array 110 may be oriented at different angles. Examples of nonplanar substrates include various types of multi-faceted surfaces, such as a pyramidal textured surface. For instance, the pyramids of the surface 114 are square-based pyramids with four sides defined by the <111> crystallographic planes. Further details regarding examples of such nonplanar substrates and corresponding dopant gradients are provided in International Publication No. WO 2021/195484 (“Doping Gradient-Based Photocatalysis,” the entire disclosure of which is hereby incorporated by reference. The manner in, or degree to, which the surface 114 is multi-faceted or otherwise nonplanar may vary. For instance, the surface 114 may have any number of faces oriented at any angle. The pyramids or other shapes along the surface 114 may be uniform or non-uniform.

The nanowires 112 may be configured to generate electron-hole pairs upon illumination. The nanowires 112 may be configured to generate the electron-hole pairs upon absorption of light at certain wavelengths (e.g., solar wavelengths). In some cases, each nanowire 112 may have multiple segments, with each segment being configured to absorb light over a respective range of wavelengths and, thus, improve the efficiency of the photocatalytic water splitting. For instance, each nanowire 112 may include a stacked or layered arrangement of semiconductor materials. Each layer in the arrangement may be configured for absorption of light of solar wavelengths (e.g., infrared, visible, and/or ultraviolet wavelengths).

The layered arrangement of semiconductor materials is used to establish a multi-band structure, such as a quadruple band structure. Each layer or segment of the arrangement may have a different semiconductor composition to establish a different bandgap. For instance, in III-nitride examples, the layers or segments of the arrangement may have different indium and gallium compositions. Examples of layered arrangements configured to provide a multi-band structure are shown and described below.

The layered arrangement of the nanowires 112 may vary from the examples described herein. For example, further details regarding the formation and configuration of multi-band structures, including, for instance, triple-band structures, are provided in U.S. Pat. No. 9,112,085 (“High efficiency broadband semiconductor nanowire devices”) and U.S. Pat. No. 9,240,516 (“High efficiency broadband semiconductor nanowire devices”), the entire disclosures of which are incorporated by reference.

The semiconductor composition of each nanowire 112 may be configured to improve the efficiency of the water splitting and CO₂ reduction reaction in additional ways. For instance, in some cases, the semiconductor composition of each nanowire 112 may include doping to promote charge carrier separation and extraction, as well as to facilitate the establishment of a photochemical diode (e.g., to promote charge carrier separation and extraction). For example, a dopant concentration of the semiconductor composition may vary laterally and/or from layer to layer.

In examples involving III-nitride compositions, the dopant may be or include magnesium. Further details regarding the manner in which magnesium doping promotes charge carrier separation and extraction are set forth in U.S. Pat. No. 10,576,447 (“Methods and systems relating to photochemical water splitting ”), the entire disclosure of which is incorporated by reference. Additional or alternative dopant materials may be used, including, for instance, silicon, carbon, zinc, and beryllium, depending on the semiconductor light absorber of choice.

The photocatalytic device 106 further includes a binary catalyst arrangement supported by the array 110 of nanowires 112. As shown in FIG. 1, each nanowire is decorated with a plurality of nanoparticles 116-118 having a binary catalyst arrangement. The binary catalyst arrangement may be composed of, or otherwise include, gold and iridium as binary catalysts. The gold and iridium may be alloyed as described herein. The nanoparticles 116-118 are distributed or disposed over the array 110 of nanowires 112. Pluralities of the nanoparticles 116-118 are disposed on each nanowire 112, as schematically shown in FIG. 1. The nanoparticles 116-118 are distributed across or along the outer surface(s) of each nanowire 112. In the example of FIG. 1, the nanoparticles 116-118 are disposed along sidewalls 120 of the nanowires 112. Alternatively or additionally, the nanoparticles 116-118 are disposed along one or more other surfaces of the nanowires 112, such as a top or upper surface.

The binary catalysts of the nanoparticles 116-118 are configured to facilitate or promote the CO₂ reduction reaction. Further details regarding the formation, configuration, functionality, and other characteristics of the nanoparticles 116-118 in conjunction with a nanowire array are set forth herein and/or in one or more of the above-referenced U.S. patents.

In some cases, the nanoparticle catalysts 116 are composed of, or otherwise include, a parental metal other than gold. For instance, additional or alternative Group IB metals may be used, including, for instance, silver and copper, as well as alloys thereof.

In some cases, the nanoparticle catalysts 116 are composed of, or otherwise include, a secondary metal other than iridium. For instance, additional or alternative platinum group metals may be used, including, for instance, ruthenium, rhodium, palladium, osmium, and platinum, as well as alloys thereof.

The binary catalyst arrangement may have a gold-to-iridium molar ratio to tune or establish one or more output product ratios for the device 100. For instance, the ratio of C₂H₆ to CH₄ and/or syngas may be tuned in this manner. In some cases, the average Au/Ir ratio falls within a range from about 0.45/0.55 to about 0.75/0.25, but other ratios may be used.

The distribution of the binary catalysts may be uniform or non-uniform. For instance, the binary catalysts may thus be distributed uniformly in the sense that each nanowire 112 is decorated with the binary catalysts. The specific location of the binary catalysts on each nanowire 112 may be differ from nanowire to nanowire. The schematic arrangement of FIG. 1 is shown for ease in illustration.

The nanowires 112 and the nanoparticles 116-118 of binary catalysts are not shown to scale in the schematic depiction of FIG. 1. The shape of the nanowires 112 and the nanoparticles 116-118 may also vary from the example shown. Further details regarding the nanowires and binary catalyst arrangement, including the fabrication thereof, are provided below.

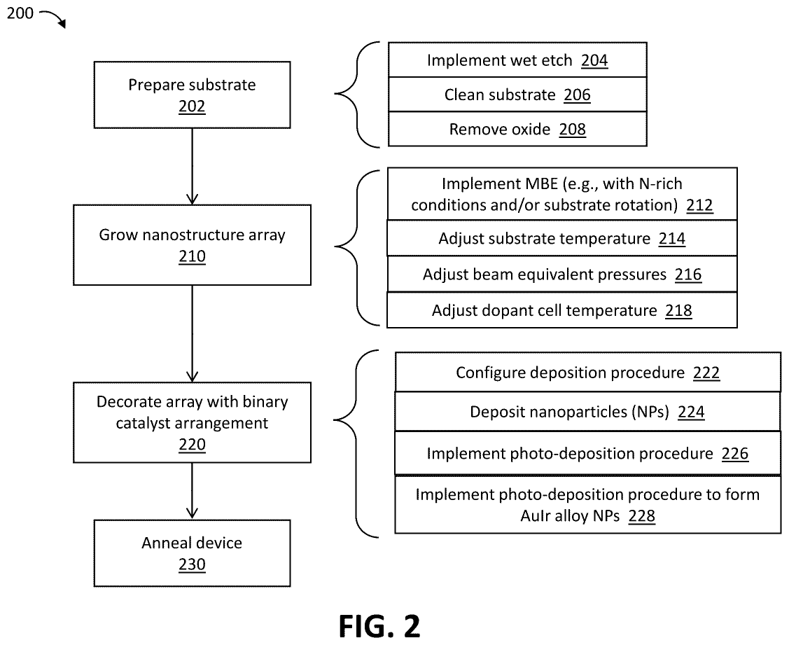

The nanowire and binary catalyst arrangement may be fabricated on a substrate (e.g., a silicon substrate) via nanostructure engineering. In one example, molecular beam epitaxial (MBE) growth of the nanowires is followed by photo- and/or other deposition of the catalysts. Further details regarding example fabrication procedures are provided below, e.g., in connection with FIG. 2.

The nanowires 112 may facilitate the water splitting in alternative or additional ways. For instance, each nanowire 112 may be configured to extract charge carriers (e.g., electrons) generated in the substrate 108 (e.g., as a result of light absorbed by the substrate 108). In such cases, the opposite side of the substrate 108 may be configured for hole extraction. The extraction brings the charge carriers to external sites along the nanowires 112 for use in the reduction of CO₂ and/or other reactions.

FIG. 2 depicts a method 200 of fabricating a photocatalytic device for CO₂ reduction in accordance with one example. The method 200 may be used to manufacture any of the devices described herein or another device. The method 200 may include additional, fewer, or alternative acts. For instance, the method 200 may or may not include one or more acts directed to annealing the device (act 230).

The method 200 may begin with an act 202 in which a substrate is prepared or otherwise provided. The substrate may be or be formed from a silicon wafer. In one example, a 2-inch Si wafer was used, but other (e.g., larger) size wafers may be used. Other semiconductors and substrates may be used.

In some cases, the act 202 includes an act 204 in which a wet or other etch procedure is implemented to define the surface (e.g., nonplanar surface). For example, the etch procedure may be or include a crystallographic etch procedure. In silicon substrate examples, the crystallographic etch procedure may be or otherwise include a KOH etch procedure. In such cases, if the substrate has a <100> orientation, the wet etch procedure establishes that the surface includes a pyramidal textured surface with faces oriented along <111> planes, but additional or alternative facets may be present in some cases.

The act 202 may include fewer, additional, or alternative acts. For instance, in the example of FIG. 1, the act 202 includes an act 206 in which the substrate is cleaned, and an act 208 in which oxide is removed.

In one example, a prime-grade polished silicon wafer is etched in 80° C. KOH solution (e.g., 1.8% KOH in weight with 20% isopropanol in volume) for 30 minutes to form the micro-textured surface with Si pyramids. After being neutralized in concentrated hydrochloric acid, the substrate surface is cleaned by acetone and/or methanol, and native oxide is removed by 10% hydrofluoric acid.

The method 200 includes an act 210 in which a nanowire or other nanostructure array is grown or otherwise formed on the substrate. Each nanowire is formed on the surface of the substrate such that each nanowire extends outward from the surface of the substrate. Each nanowire has a semiconductor composition, as described herein. The nanowire growth may be achieved in an act 212 in which molecular beam epitaxy (MBE) is implemented. The MBE procedure may be implemented under nitrogen-rich conditions. Alternatively or additionally, the substrate may be rotated during the MBE procedure such that each nanostructure is shaped as a cylindrically shaped nanostructure. Each nanowire may thus have a circular cross-sectional shape, as opposed to a plate-shaped or sheet-shaped nanostructure.

In some cases, the MBE procedure may be modified to fabricate the arrangement of layers or segments of each nanowire directed to providing a multi-band structure. Various parameters may be adjusted to achieve the different composition levels of the layers. For instance, the substrate temperature may be adjusted in an act 214. Beam equivalent pressures may be adjusted in an act 216. In some cases, a dopant cell temperature is adjusted to control the doping (e.g., Mg doping) of the nanowires.

In one example, Mg-doped InGaN nanowires were grown by plasma-assisted molecular beam epitaxy (MBE) under N-rich conditions. The growth parameters included a gallium (Ga) beam equivalent pressure of about 7E-8 Torr, a nitrogen flow rate of 1 sccm, and a plasma power of 350 W. The substrate temperature, indium (In) beam equivalent pressure (BEP), and magnesium (Mg) cell temperature were tuned to synthesize different single-band or multi-band InGaN nanowires with various p-doping and alloy concentrations. For instance, for single-band p-GaN nanowires or a GaN layer of a multi-band structure, the substrate temperature was 685 ° C., and Ga BEP was about 7E-8 Torr. The p-type doping level was tuned by using different Mg cell temperatures. For a p-In_{0.20}Ga_{0.80}N nanowire layer, the substrate temperature was 675° C., the Ga BEP was about 7E-8 Torr, and the In BEP was about 7.3E-8. For p-In_{0.27}Ga_{0.73}N nanowire layers, the substrate temperature was 662° C., the Ga BEP was about 7E-8 Torr, and the In BEP was about 7.3E-8. For p-In_{0.35}Ga_{0.65}N nanowire layers, the substrate temperature was 640° C., the Ga BEP was about 7E-8 Torr, and the In BEP was about 3.5E-8. For quadruple-band InGaN nanowires, the growth conditions are similar to those of the constituting single-band nanowires but with varying thicknesses for each segment. The substrate temperature may refer to a thermocouple reading of a substrate heater, which may be different from the actual substrate surface temperature, which may depend on the sample size, substrate holder, and mounting configuration.

The act 210 may include additional, fewer, or alternative acts. For instance, the act 210 may include one or more acts directed to forming a seed other initial layer in preparation for growth of the nanowires. The seed layer may be configured to promote the nucleation of the nanowires. In some cases, the seed layer is composed of, or otherwise includes, Ga. Further details regarding the use of seed layers are set forth below in connection with a number of examples as well as in the above-referenced patent documents.

As shown in FIG. 2, the method 200 further includes an act 220 in which the array is decorated with a binary catalyst arrangement. Binary catalysts are deposited across the array of nanowires. As described herein, the binary catalysts may be disposed in an alloyed configuration.

The selective deposition of the nanoparticles may be achieved via implementation of a deposition procedure that concurrently provides the parental and secondary metals. In the example of FIG. 2, the act 220 includes an act 222 in which the deposition procedure is configured. The configuration may be directed to establishing a ratio of the binary catalysts. For instance, the deposition procedure(s) may be configured to establish a gold-to-iridium ratio. Further details are provided below in connection with a number of examples.

In the example of FIG. 2, the act 220 include an act 224 in which nanoparticles are deposited on the nanowires. The nanoparticles may be composed of, or otherwise include, gold and iridium and/or other parental and secondary metals, as described herein. In some cases, the deposition of the nanoparticles includes implementation of a photo-deposition procedure to deposit the parental and secondary metals in an act 226. For instance, a photo-deposition procedure may be implemented in an act 228 to form AuIr alloyed nanoparticles. Additional or alternative procedures may be used, including, for instance, e-beam, thermal or other physical vapor deposition procedures, such as sputtering, as well as atomic layer deposition procedures. Further details regarding examples of the photo-deposition procedures are set forth hereinbelow and in one or more of the above-referenced U.S. patents.

The method 200 may include one or more additional acts directed to forming the photocatalytic structures of the device. For instance, in some cases, the method 200 includes an act 230 in which the photocatalytic structures of the device are annealed. The parameters of the anneal process may vary.

The order of the above-described acts of the method 200 may differ from the example shown. For instance, the annealing of the act 230 may be implemented before or after the deposition of the nanoparticles in the act 220.

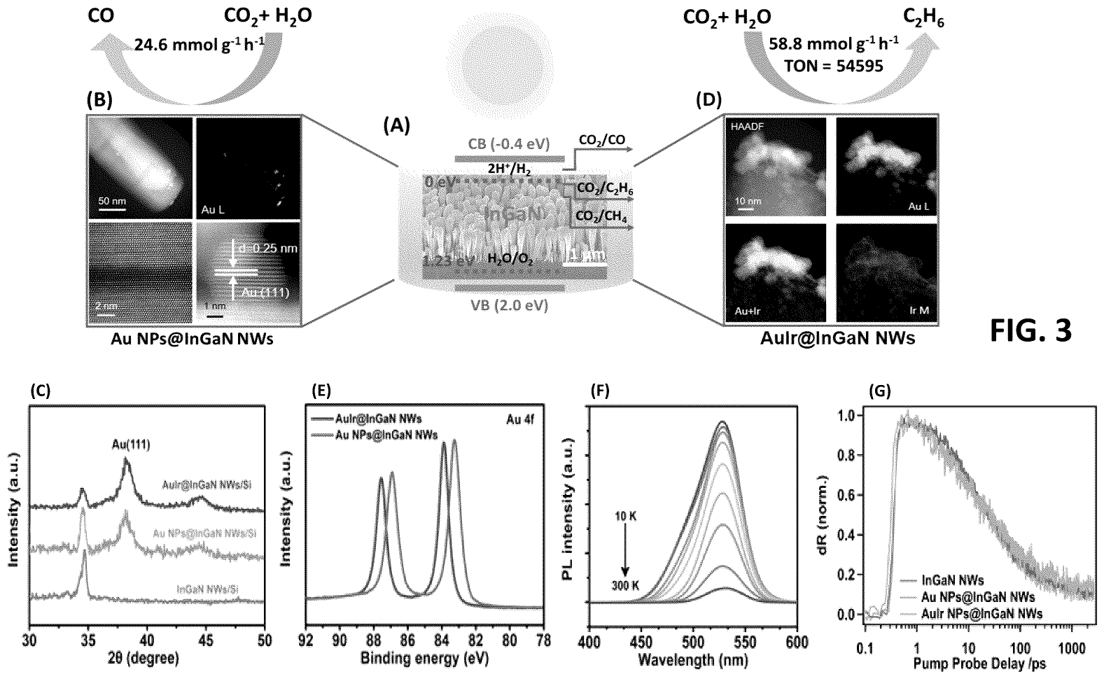

Details regarding examples of the above-described devices, and methods are now provided in connection with FIGS. 3-6. The examples include a photocatalytic device having AuIr-decorated InGaN/GaN nanowire structures for use in CO₂ reduction to one or more multi-carbon products.

Growth and Fabrication of Example Photocatalytic Devices. Vertically Aligned InGaN/GaN nanowires were grown on a Si substrate by radio frequency plasma-assisted MBE under nitrogen-rich conditions. To achieve efficient harvesting of solar photons, a multi-stacked nanowire structure of GaN (Mg-doped)-InGaN (Mg-doped) segments was grown. Detailed descriptions of the growth procedures can be found in the above-referenced patent publications, as well as in Kibria et al. Visible light-driven efficient overall water splitting using p-type metal-nitride nanowire arrays. Nat. Commun. 6, 6797 (2015), the entire disclosure of which is incorporated by reference.

In the example of FIG. 3, InGaN nanowires were epitaxially grown on a 3-inch silicon wafer as described in the above-referenced publications, followed by photo-depositing binary catalysts (or co-catalysts) of Au and Ir. The conduction band (CB) and valence band (VB) edges of the InGaN nanowires/Si structure shown in FIG. 3, part A, are well positioned with respect to the redox potentials for CO₂ reduction with water, thermodynamically making applied bias-free C₂₊ compounds synthesis without sacrificial agents possible. As characterized by scanning electron microscopy, the InGaN nanowires are vertically aligned on the silicon substrate and possess an average height of 600-750 nm with varied lateral sizes from 50 to 150 nm at the top region. The loaded cocatalysts do not affect the morphologies of InGaN nanowires significantly.

High angle annular dark field-scanning transmission electron microscopy (HAADF-STEM) and elemental mapping images in FIG. 3, part B, show that the deposited Au nanoparticles on the surface of InGaN illustrate a high-quality crystal structure. The interplanar spacing lattice of d=0.25 nm is attributed to Au (111), which can be observed at 2θ of 38.2° by X-ray diffraction measurement (FIG. 3, part C).

A series of examples having binary AuₓIr₁₋ₓ nanoparticles were formed by coupling with secondary iridium using Au as the parental metal, where x represents the molar ratio of gold.

The introduction of iridium did not affect the crystal structure of Au (111). Furthermore, as characterized by Auger electron spectroscopy (AES), Au/Ir ratios of the examples were estimated to be 0.55/0.45, 0.44/0.56, and 0.26/0.74, respectively. As shown in FIG. 3, part D, the as-designed AuₓIr₁₋ₓ binary catalytic system features a bimetallic solid structure.

By energy dispersive spectrometer (EDS) quantification, the Au/Ir ratio on the surface of the AuIr nanoparticles is approximately 51/49 while the center (or internal) region illustrates a higher proportion of Au at 81%. The AuIr ratio may thus vary across each nanoparticle in the array. Accordingly, general references to the ratio refer to the average ratio across the entire nanoparticle unless otherwise stated.

The total loading amount of Au and Ir in an Au_{0.44}Ir_{0.56}/InGaN nanowire example was evaluated to be 0.012μmol·cm⁻² by inductively coupled plasma-atomic emission spectroscopy (IPC-AES). X-ray photoelectron spectroscopy (XPS) characterization was conducted to identify the elemental oxidation states of the artificial photosynthesis integrated devices. It is observed that the featured peak of Au 4f_{7/2} in an AuIr decorated InGaN nanowire/Si example appears at 83.9 eV, which is positively shifted by +0.6 eV compared to that of Au 4f_{7/2} at 83.3 eV in an example having only Au nanoparticles (see FIG. 3, part E). This finding validates the electronic interaction between Au and Ir in an AuIr-decorated InGaN nanowire example. It may increase the likelihood of C—C coupling by influencing the catalytic properties of the binary arrangement, which is discussed further below.

Aside from the catalysts, the semiconductor material of the nanowires also plays a useful role in the photocatalytic CO₂ reduction reaction by supplying charge carriers with necessary redox potentials to drive the reactions. For instance, the radiative recombination efficiency of charge carriers of the InGaN nanowires was measured by temperature-dependence photoluminescence (TD-PL) spectroscopy (FIG. 3, part F). The internal quantum efficiency calculated using the integrated intensity ratio of the PL spectra recorded at 300 K and 10 K³⁴, i.e., I_{300 K}/I_{10 K} was about 5%. The measured internal quantum efficiency signified a low radiative recombination efficiency of the photoexcited electrons and holes in the InGaN nanowires.

Further considering the nearly defect-free crystal structure of InGaN nanowires, the likelihood of the photoexcited charge carriers for participating chemical reactions is enhanced, thus favoring high-efficiency carbon dioxide reduction. Generally, as the CO₂ reduction reaction product distribution varies as a function of applied potential in electrocatalysis, using tuned PL quantum efficiency may yield tailored CO₂ reduction reaction product distributions for these bias-free photocatalysts. Furthermore, in the disclosed examples, the nanowire radius (e.g., about 25-75 nm) is smaller than the carrier diffusion length in crystalline Mg-doped p-type GaN (93±7 nm at 295 K). Therefore, charge carrier extraction to the lateral surfaces of the nanowires is extremely efficient, regardless of the presence of metal particles or not, which is highly favorable for the reaction. It is well verified by the transient reflection spectroscopy measurements on the various samples (FIG. 3, part G). Moreover, the well-defined 1-D morphology of the InGaN nanowires is highly favorable for absorbing photons by minimizing light reflection. Together, the AuIr decorated GaN nanowire examples demonstrate highly efficient photocatalytic CO₂ reduction due to the outstanding optical, electronic, and catalytic properties of the examples.

The photocatalytic performance of the example devices is now described in comparison with a device structure having no catalysts and a device structure having only Au nanoparticles. The reactions were carried out in a glass chamber in CO₂ aqueous solution under concentrated light illumination of 3.5 W cm⁻² without any applied bias or sacrificial agents.

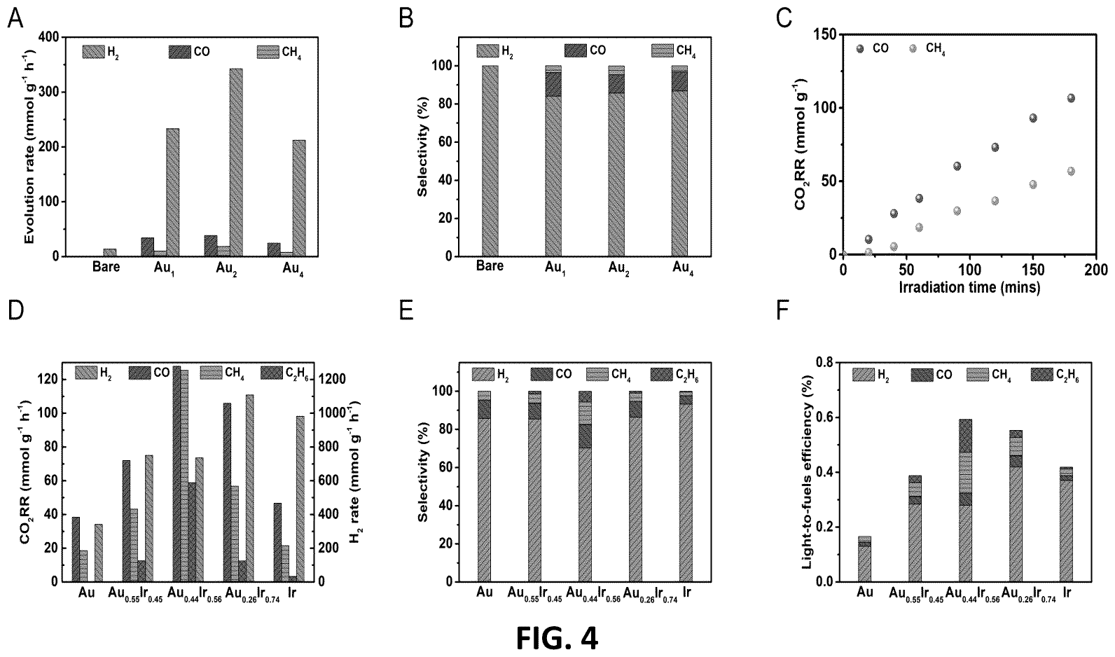

Without any catalysts, InGaN nanowires showed little hydrogen evolution activity without any carbon products. With the decoration of various amounts of only Au, InGaN nanowires on Si exhibited considerable activity of CO₂ reduction (see FIG. 4, parts A and B). C₁ compounds are favorably produced by gold alone; and CO is the major carbon product. The activity and selectivity largely depended on Au nanoparticle load and/or size. The formation rate of CO increases with Au loading and reaches a peak value of 38.4 mmol g⁻¹ h⁻¹ with a selectivity of 9.6% at an average 6.0 nm of Au nanoparticles. In this case, the hydrogen evolution reaction is much more favored compared to CO₂ reduction and the selectivity of H₂ is as high as 85.7%, further suggesting the challenge of selective conversion of CO₂. Reduced performance of CO (24.6 mmol g⁻¹ h⁻¹) is observed at a higher Au loading, probably originating from the decreased catalytic activity as the size of Au nanoparticles further increases up to 10.4 nm.

To gain an overall view on the reaction involving pure Au, the evolution of CO₂ reduction products over the optimized Au₂ decorated InGaN nanowires on Si was tested as a function of the illumination time. Illustrated in FIG. 4, part C, CO is dominantly yielded, accompanying with CH₄ evolved as the byproduct. Across the time range examined, C₂₊ compounds were not detected over pure gold, verifying that Au is not capable of catalyzing C—C coupling toward C₂₊ compounds.

In contrast, the performance of an example device with AuIr catalyst-decorated InGaN nanowires on Si is substantially different from that of pure Au nanoparticles on InGaN nanowires (see FIG. 4, parts D and E). The overall activity of various products is significantly improved by the addition of Ir. More importantly, AuIr decorated InGaN nanowires demonstrated C₂H₆ synthesis activity. In terms of the activity of CH₄ and C₂H₆, the example device with AuIr decorated InGaN nanowires compared favorably with previously reported photocatalytic devices. Most of the previously reported photocatalytic devices only showed low CH₄ activity on the order of μmol g⁻¹ h⁻¹ and were almost not active for C₂₊ compounds synthesis. In stark contrast, a C₂H₆ evolution rate of 58.8 mmol g⁻¹ h⁻¹ was achieved by an example device having Au_{0.44}Ir_{0.56} on InGaN nanowires with a selectivity of 5.6%. Meanwhile, the formation rate of CH₄ is as high as 125.4 mmol g⁻¹ h⁻¹, which is several orders of magnitude higher than that of the state-of-the-art catalytic systems. The total selectivity of hydrocarbons including CH₄ and C₂H₆ reached up to 17.6%.

It is worth emphasizing that a mixture of CO and H₂, an important chemical feedstock named syngas, was also formed as useful byproducts with appreciable activity of 863.4 mmol g⁻¹ h⁻¹ (H₂=735.6, CO=127.8) and high selectivity of 82.4%. Taking all the fuels into consideration, a benchmarking LTFs efficiency of 0.59% is achieved by the example device having Au_{0.44}Ir_{0.56} decorated InGaN nanowires on Si (FIG. 4, part F), which is 3.5 times higher than that of pure Au nanoparticle decorated InGaN nanowires on Si (0.17%).

What is more, Ir decorated InGaN nanowires were also active for C₂H₆ formation, albeit with much lower activity (3.3 mmol g⁻¹ h⁻¹) and lower selectivity (0.31%) than that of the example device having AuIr decorated InGaN nanowires. These findings validate that Ir is useful for C—C bond formation.

Control experiments established that the reaction did not occur if AuIr was directly deposited on silicon because silicon was not able to produce energetic charge carriers for CO₂ reduction owing to its narrow bandgap. It follows that the combination of both AuIr and InGaN nanowires is useful for CO₂-to-C₂H₆ conversion.

The influence of light intensity on the reaction was also tested. Under relatively weak light intensity varied from 0.1 W cm⁻² to 0.5 W cm⁻², the photocatalytic activity of the example device was low. It could be attributed to the insufficient photogenerated electron-hole pairs. In contrast, under high light intensity above 1.5 W cm⁻², the efficiency of CO₂ reduction was sharply increased by the continuously increasing light intensity. Under concentrated illumination of 3.5 W cm⁻², InGaN nanowires are photoexcited to produce abundant electrons and holes. The photogenerated electrons then migrate to the AuIr deposited on the nanowires for carbon dioxide reduction and hydrogen evolution, while the holes will be consumed by water oxidation.

The photothermal-assisted effect of the concentrated light illumination on the reaction was also investigated. As measured by a thermocouple, the increasing light intensity led to an enhanced heating rate of the reaction system because of the photothermal effect. Moreover, it is found that under three unvaried concentrated light illumination at 1.5 W/cm², 2.5 W/cm², and 3.5 W/cm², respectively, the activity was increased with the water bath temperature set from 20° C. to 50° C. by an external temperature-control system. The further increasing temperature however did not show an apparent enhancement in the performance, which is probably due to the lowered CO₂ solubility in water under higher temperature. Meanwhile, under ultraviolet light illumination, the architecture exhibited only moderate photocatalytic CO₂ reduction activity. In stark contrast, the architecture was hardly active for photocatalytic CO₂ reduction illuminated by infrared light because the infrared light is not capable of exciting the InGaN nanowires to produce photogenerated charge carriers. These results indicate that the reaction proceeds via thermal-assisted photocatalysis, and the photogenerated charge carriers play a useful role in the superior performance. It is also noted that in the absence of CO₂, no carbon-derived products except for hydrogen were detected from photoreduction by the example device having Au_{0.44}Ir_{0.56} decorated InGaN nanowires on Si under vacuum without varying other reaction conditions, e.g., light intensity of 3.5 W·cm⁻².

To further verify the source of the C₂₊ photoreduction products, an isotopic test was conducted by feeding ¹³C-labeling CO₂. It was observed that the typical feature of ¹³C₂H₆ was detected by gas chromatography-mass spectroscopy, providing solid evidence that the C₂₊ products originated from CO₂ reduction. The oxidation reaction, which is entailed in the entire artificial photosynthesis, was also tested. By gas chromatography, it was observed that, although not stoichiometric, oxygen was produced from the oxidation half reaction, in concurrent formation with H₂, CO, CH₄, and C₂H₆ from the reduction half reaction side. Meanwhile, the formation of H₂O₂ from water oxidation was also detected by using aqueous potassium permanganate solution. Furthermore, control testing showed that, under high intensity ultraviolet light, O₂ may be consumed in connection with forming other oxygen species, such as singlet oxygen. Together, the test results described above establish the implementation of an artificial photosynthesis process of photocatalytic CO₂ reduction toward various products via an example device having AuIr decorated InGaN nanowires on Si, along with concurrent water oxidation into H₂O₂, O₂ and other oxygen species.

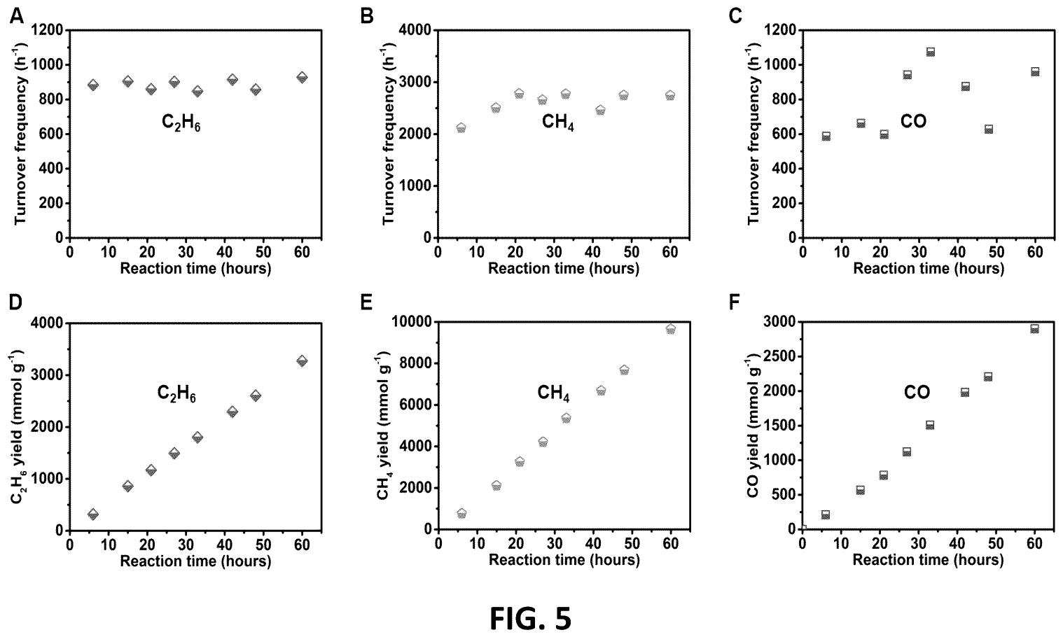

To further evaluate the intrinsic activity of the example device having Au_{0.44}Ir_{0.56} decorated InGaN nanowires on Si, turnover frequency (TOF), turnover number (TON), evolution rates, and yields of various products from CO₂RR were measured. Under 60 hours of illumination, TOF of C₂H₆ did not vary very much at an average value of 910 h⁻¹ (FIG. 5, part A), and TON reached a record-high value of 54,595. A total C₂H₆ yield of 3,275.7 mmol g⁻¹ was achieved with an average rate of 54.6 mmol g⁻¹ h⁻¹ (FIG. 5, part D). Both CH₄ and CO exhibited a similar tendency to C₂H₆. The TOF of CH₄ shown in FIG. 5, part B, was sustained at a high value of 2,681 h⁻¹ whilst the TOF of CO exhibited relatively wide variations (FIG. 5, part C). Meanwhile, CH₄ was produced at an average rate of 160.9 mmol g⁻¹ h⁻¹ with a total yield of 9,653.1 mmol·g⁻¹. Such high performance is attributed to the synergism of high optical absorption, high charge carrier extraction efficiency, as well as unique catalytic properties of the components of the example device. The morphology remained nearly intact after the long-term testing. Furthermore, as measured by ICP-AES, there was no observation in the elemental In and Ga in the post-reaction media, indicating high stability of the epitaxial InGaN nanowires. The N-rich surface, together with the ionic bonds of the III-nitride material, function as a protection layer against photo-corrosion. However, the harsh operation conditions, e.g., high light intensity, led to Au and Ir falling off from the nanowires after a long-term operation, which was also validated by IPC-AES measurement. Such decoupling of the AuIr catalysts and the nanowires may thus result in the degradation of the photocatalytic activity.

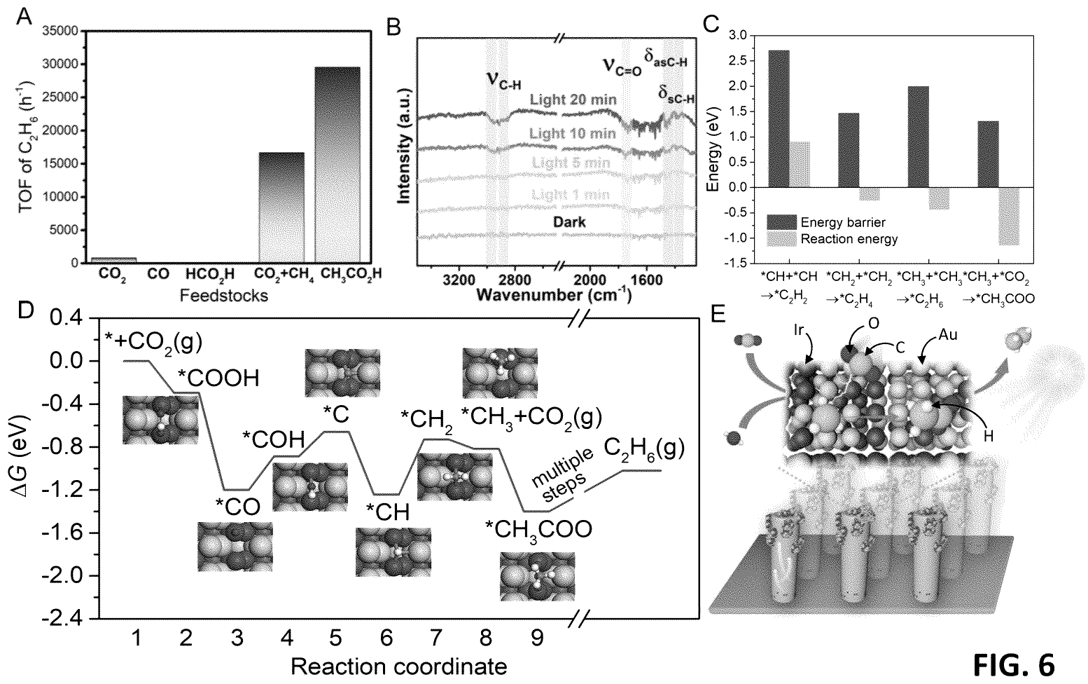

To gain more insight into the performance of the example device, the TOF values of C₂H₆ of various feedstocks, e.g., CO₂, CO, HCOOH, CH₃COOH, and CH₄+CO₂, were estimated in distilled water over the Au_{0.45}Ir_{0.55} decorated InGaN nanowires on Si under the same reaction conditions (FIG. 6, part A). Because CO dimerization is widely recognized as a step of C—C coupling during CO₂ reduction, CO was first used as feedstock. Surprisingly, it was found that the example device having Au_{0.44}Ir_{0.56} decorated InGaN nanowires on Si is not active for catalyzing C₂H₆ synthesis in the presence of CO, which is the same as HCOOH. In stark contrast, the TOF of C₂H₆ was significantly improved by a factor of 35.5 using CH₃COOH as the feedstock, approaching to 29543 h⁻¹. Meanwhile, C₂H₆ demonstrated a nearly 20-fold enhancement in TOF when CH₄ was introduced into CO₂. These results exclude the possibility that C₂H₆ is formed from C—C coupling via the dimerization of CO.

To uncover the reaction mechanism, in-situ diffuse reflectance infrared Fourier-transform spectroscopy (DRIFTS) was employed to investigate the CO₂ reduction at the molecular level. It was found that, all the featured signals over the example device having Au_{0.44}Ir_{0.56} decorated InGaN nanowires on Si are strengthened with the irradiation time over the period of 0 to 20 mins (FIG. 6, part B), while pure Au on InGaN nanowires did not exhibit featured peaks from CO₂ reduction because of the weak interaction between Au and the reactants/intermediates. In particular, the peaks at 1750 cm⁻¹ are associated with the stretching model of the adsorbed C═O species (ν_{C═O}). Meanwhile, the double peaks at the range of 2700 to 3000 cm⁻² are related to the vibrational absorption of C—H (ν_{C—H}) while the peaks at 1450 and 1370 cm⁻¹ originate from the deformation of the vibration model of δ_{C—H}. Based on these observations, it appears that the key intermediate of C₂H₆ contains —CHₓ and C═O groups, and the insertion of CO₂ into a methyl group is a step in the C—C coupling, which has been observed during CH₄ reforming with CO₂ over a zinc-doped cerium catalyst.

To provide insight into the mechanism of C₂H₆ formation, density functional theory (DFT) calculations were conducted over four different surface compositions (AuₓIr_{y}) on (111) facet, i.e., Au₄₋ₓIrₓ (x=0, 1, 2, and 3) alloys. The reaction energies (ΔG) of multiple elementary steps and their bifurcate routes were estimated before C—C coupling occurs. Au₂Ir₂ exhibits the minimum value for the potential-determining step (PDS), which is the *CH protonation to *CH₂, among the screened Au—Ir compositions. Au₂Ir₂ was focused on via further DFT calculations on the reaction mechanism of direct C—C coupling by considering *CH+*CH→*C₂H₂, *CH₂+*CH₂ →*C₂H₄, *CH₃+*CH₃→C₂H₆(g), and CO₂ insertion into *CH₃ to *CH₃COO. The insertion of CO₂ into *CH₃ toward *CH₃COO exhibited the lowest reaction energy and energy barrier simultaneously (FIG. 6, part C), thereby being the possible mechanism of C—C coupling for C₂H₆ synthesis. These theoretical observations are consistent with the operando spectroscopy measurements and feedstock control experiments. The TOF of C₂H₆ using CH₂OHCOOH as the feedstock is about 89.2 h⁻¹, which is 331 times lower than that using CH₃COOH as the intermediate. In addition, the free energy diagram of the reaction pathway before C—C coupling on Au₂Ir₂(111) facet is summarized in FIG. 6, part D, i.e., CO₂ first adsorbs and is hydrogenated into COH (other than *CHO), and then *COH will be further hydrogenated into *C, *CH, *CH₂, and *CH₃, and finally, another *CO₂ inserts the formed *CH₃ intermediate to generate the *CH₃COO intermediate. The Ir sites in the Au—Ir alloys increase CO₂ reduction activity by lowering the reaction energy of key elementary steps (e.g., CO₂ to *COOH on pure Au and *CO to *CHO on Au₃Ir₁ alloy) and steer the selectivity from dominant hydrogen evolution reaction to C—C coupling with the optimal value achieved around Au:Ir ratio of 1. The synergy between gold and iridium for enhancing CO₂ reduction is decreased if a large portion of Ir is added into Au (e.g., Au_{0.24}Ir_{0.76}). Based on the feedstock experiments, operando spectroscopy measurements, and theoretical calculations above, gold is regulated, manipulated, or otherwise mediated by iridium to achieve distinct C—C coupling by insertion of CO₂ into *CH₃, thus facilitating unassisted C₂H₆ synthesis from CO₂ and H₂O over the AuIr decorated InGaN nanowires on Si (see, e.g., FIG. 6, part E).

The above-described testing of the example device establishes that the unique mediation of gold by iridium is capable of breaking the bottleneck of C—C coupling during light driven CO₂ reduction. The assembly of AuIr with InGaN nanowires thus supports C₂ alkane synthesis from CO₂ and H₂O without using any external bias or any sacrificial agents, as a result of the distinct catalytic properties of AuIr together with the high optical absorption, and high charge charrier efficiency of the InGaN nanowires.

Described above are examples of the synthesis of C₂₊ compounds using sunlight, carbon dioxide, and water as the only inputs. The disclosed devices provide examples that break the bottleneck of C—C coupling and realize a useful artificial photosynthesis integrated device. The examples demonstrate that gold, mediated by iridium, is capable of catalyzing the reduction of CO₂, achieving C—C coupling by insertion of CO₂ into —CH3. Owing to a combination of optoelectronic and catalytic properties, the assembly of the binary AuIr catalyst with InGaN nanowires vertically aligned on a silicon substrate (AuIr decorated InGaN nanowires on Si) achieved a C₂H₆ activity of 58.8 mmol g⁻¹ h⁻¹ with a remarkable turnover number of 54,595 over 60 hours. A light-to-fuels efficiency of ˜0.59% for solar fuels production from CO₂ and H₂O was achieved under concentrated light illumination without any applied bias or any sacrificial agents. The disclosed devices thus present an artificial photosynthesis integrated device for unassisted C₂₊ compounds synthesis from CO₂ and H₂O.

The term “about” is used herein in a manner to include deviations from a specified value that would be understood by one of ordinary skill in the art to effectively be the same as the specified value due to, for instance, the absence of appreciable, detectable, or otherwise effective difference in operation, outcome, characteristic, or other aspect of the disclosed methods and devices.

The present disclosure has been described with reference to specific examples that are intended to be illustrative only and not to be limiting of the disclosure. Changes, additions and/or deletions may be made to the examples without departing from the spirit and scope of the disclosure.

The foregoing description is given for clearness of understanding only, and no unnecessary limitations should be understood therefrom.

What is claimed is:

1. A photocatalytic device comprising:
a substrate; and
an array of conductive projections supported by the substrate and extending outward from the substrate, each conductive projection of the array of conductive projections having a semiconductor composition configured for charge carrier generation in response to light radiation;
wherein each conductive projection of the array of conductive projections is decorated with a catalyst arrangement, the catalyst arrangement comprising a parental Group IB metal and a secondary platinum group metal.

2. The photocatalytic device of claim 1, wherein:
the parental Group IB metal is gold; and
the secondary platinum group metal is iridium.

3. The photocatalytic device of claim 1, wherein the catalyst arrangement is a binary catalyst arrangement.

4. The photocatalytic device of claim 1, wherein the catalyst arrangement comprises an alloy of gold and iridium.

5. The photocatalytic device of claim 1, wherein the catalyst arrangement has a parental-to-secondary metal ratio that falls in a range about 0.45/0.55 to about 0.75/0.25.

6. The photocatalytic device of claim 1, wherein the semiconductor composition comprises a III-nitride semiconductor material.

7. The photocatalytic device of claim 6, wherein the III-nitride semiconductor material is InGaN.

8. The photocatalytic device of claim 1, wherein each conductive projection of the array of conductive projections comprises a nanowire.

9. The photocatalytic device of claim 1, wherein:
each conductive projection of the array of conductive projections comprises a layered arrangement of semiconductor materials; and
the layered arrangement of semiconductor materials establishes a multiple band structure.

10. The photocatalytic device of claim 1, wherein the catalyst arrangement is configured for catalysis of carbon dioxide (CO₂) reduction.

11. A method of using the photocatalytic device of claim 1, the method comprising:
illuminating the photocatalytic device with incident light radiation; and
capturing a product of the CO₂ reduction.

12. The method of claim 11, wherein the catalyst arrangement is configured such that the product comprises C₂H₆.

13. The method of claim 11, wherein the photocatalytic device is illuminated without application of a bias voltage to the photocatalytic device.

14. The method of claim 11, further comprising:
disposing the photocatalytic device in a container; and
supplying water or water vapor and CO₂ to the container.

15. The method of claim 11, wherein illuminating the photocatalytic device is implemented while the container is free of a sacrificial agent for the CO₂ reduction.

16. A method of fabricating a photocatalytic device, the method comprising:
providing a substrate having a surface;
forming an array of conductive projections on the substrate such that each conductive projection of the array of conductive projections extends outward from the substrate, each conductive projection of the array of conductive projections having a semiconductor composition configured for charge carrier generation in response to light radiation; and
decorating each conductive projection of the array of conductive projections with a catalyst arrangement,
wherein decorating each conductive projection comprises concurrently depositing a parental Group IB metal and a secondary platinum group metal.

17. The method of claim 16, wherein decorating each conductive projection comprises configuring a deposition procedure to establish a parental metal-to-secondary metal ratio that falls in a range about 0.45/0.55 to about 0.75/0.25.

18. The method of claim 16, wherein depositing the parental Group IB metal and the secondary platinum group metal comprises implementing a photo-deposition procedure.

19. The method of claim 16, wherein forming the array of conductive projections comprises implementing a molecular beam epitaxy (MBE) procedure to grow a stack of a plurality of III-nitride semiconductor segments, wherein:
each III-nitride semiconductor segment of the plurality of III-nitride semiconductor segments has a respective bandgap for charge carrier generation in response to solar radiation; and
the stack comprises a plurality of GaN segments, each GaN segment of the plurality of GaN segments being disposed between a respective adjacent pair of III-nitride semiconductor segments of the plurality of III-nitride semiconductor segments.

20. A catalytic device comprising:
a substrate; and
a catalyst arrangement supported by the substrate, the catalyst arrangement establishing a nitride/catalyst interface of the catalytic device;
wherein the catalyst arrangement comprises a parental Group IB metal and a secondary platinum group metal.

21. The catalytic device of claim 20, wherein:
the parental Group IB metal is gold; and
the secondary platinum group metal is iridium.

22. The catalytic device of claim 20, further comprising a structure supported by the substrate, wherein the structure comprises a nitride surface, the nitride surface being decorated with the binary catalyst arrangement.

23. The catalytic device of claim 20, wherein the binary catalyst arrangement is configured to catalyze CO₂ conversion into multi-carbon products.

24. A system comprising the catalytic device of claim 20 and a thermochemical cell in which the catalytic device is disposed.

25. A system comprising the catalytic device of claim 20 and an electrochemical cell in which the catalytic device is disposed.

26. A catalytic device comprising:
a substrate; and
a binary catalyst arrangement supported by the substrate and configured for CO₂ reduction,
wherein the binary catalyst arrangement comprises gold and iridium.

27. A system comprising the catalytic device of claim 26 and a thermochemical cell in which the catalytic device is disposed.

28. A system comprising the catalytic device of claim 26 and an electrochemical cell in which the catalytic device is disposed.
